(19) United States
(12) Patent Application Publication
(10) Pub. No.: US 2011/0286950 A1
(43) Pub. Date: Nov. 24, 2011

(54) COSMETIC COMPOSITIONS OF VARYING VISCOELASTICITY

(75) Inventors: Hy Si Bui, Piscataway, NJ (US); Susan Halpern, Paramus, NJ (US); Mohamed Kanji, Edison, NJ (US)

(73) Assignee: L'OREAL S.A., PARIS (FR)

(21) Appl. No.: 13/139,846

(22) PCT Filed: Dec. 16, 2009
(86) PCT No.: PCT/US09/68251

§ 371 Date: Aug. 10, 2011

Related U.S. Application Data
(60) Provisional application No. 61/122,812, filed on Dec. 16, 2008.
(60) Provisional application No. 61/122,810, filed on Dec. 16, 2008.

(51) Int. Cl. A61K 8/81; A61Q 3/00; A61Q 19/00; A61K 31/785; A61Q 5/00

(52) U.S. Cl. 424/61; 424/70.17; 424/78.03

(57) ABSTRACT

The present invention is directed to stable cosmetic composition which is waterproof, comfortable and has a unique cushiony/bouncy texture and feel containing: (a) a reaction product of (i) at least one polyamine and (ii) at least one oil soluble high carbon polar modified polymer; (b) water; (c) at least one non-volatile solvent capable of solubilizing the polar modified polymer; and (d) optionally, at least one volatile solvent other than water,—and (e) optionally, at least one colorant.

### FIELD OF THE INVENTION

The present invention generally relates to a novel composition capable of possessing varying degrees of viscoelasticity. More particularly, the present invention relates to a composition in a gel-like form having a unique cushiony/bouncy texture and feel.

### BACKGROUND OF THE INVENTION

Many compositions, especially cosmetic compositions, have been developed for easy and comfortable application onto a targeted substrate. Unfortunately, many of these compositions are in fact difficult to apply and do not possess a smooth feel upon application. Moreover, compositions often times have a tendency to feel tacky, yielding poor application and spreadability characteristics.

In general, a gel-like texture is typically obtained with the use of expensive silicone elastomers which are swelled in a solvent. The present invention does not require the use of silicone elastomers in order to achieve the desired gel-like texture. Moreover, silicone elastomers can also be difficult to formulate with due to their chemical make up, and the gelled compositions they form may be unstable, as is, or sensitive to added ingredients.

Similarly, a rigid texture is typically obtained through the use of waxes and wax-like ingredients. The present invention, however, does not require the use of these types of conventionally-employed ingredients in order to formulate compositions having a rigid texture. On the contrary, essentially the same ingredients used to formulate a composition having a more liquid texture can be used to formulate one having a more rigid texture. Significant cost reductions are thus realized due to the relatively inexpensive cost of the ingredients used, as well as the ease in formulating such compositions.

Therefore, it is an object of the present invention to provide a gel-like composition possessing a unique cushiony/bouncy texture and feel without the need for having to use expensive ingredients and/or processing techniques, and which can serve as a stable base/matrix for the incorporation of various types of ingredients.

### BRIEF SUMMARY OF THE INVENTION

The present invention relates to a cosmetic composition comprising: (a) at least one polyamine; (b) at least one oil soluble high carbon polar modified polymer; (c) water; (d) at least one non-volatile solvent capable of solubilizing the polar modified polymer; and (e) optionally, at least one volatile solvent other than water; and (f) optionally, at least one colorant.

The present invention also relates to a cosmetic composition comprising: (a) a reaction product of (i) at least one polyamine and (ii) at least one oil soluble high carbon polar modified polymer; (b) water; (c) at least one non-volatile solvent capable of solubilizing the polar modified polymers; and (d) optionally, at least one volatile solvent other than water; and (e) optionally, at least one colorant.

The present invention relates to a cosmetic composition made by combining ingredients comprising: (a) at least one polyamine; (b) at least one oil soluble high carbon polar modified polymer; (c) water; (d) at least one non-volatile solvent capable of solubilizing the polar modified polymer; and (e) optionally, at least one volatile solvent other than water; and (f) optionally, at least one colorant.

Still another aspect of the present invention is directed to a method of making up a keratinous substrate comprising applying the above-disclosed compositions onto the substrate.

It has been surprisingly discovered that the use of the above-disclosed composition, when applied onto a keratinous substrate, delivers a combination of comfort, stability, and cushiony/bouncy texture and feel, in the absence of expensive silicone elastomers, and in an environmentally-friendly manner, while at the same time being waterproof and not requiring the presence of silicone film formers, gelling agents or emulsifiers.

It has also been surprisingly discovered that depending on the amount of water used to dissolve the polyamine present in the above-disclosed composition, the composition can take a form ranging from liquid (if there is less water) to rigid gel (if there is more water) and possesses a unique texture and feel, in the absence of use of expensive ingredients and/or processing techniques, and in a more environmentally-friendly manner. The resultant composition can be used as a stable matrix for carrying desirable ingredients to be applied in a comfortable, smooth, easily spreadable manner.

### BRIEF DESCRIPTION OF THE DRAWING

FIG. 1. depicts the effect of PEI concentration and water contents to the instantaneous creep strain oscillation of the bouncy gels from Examples 1-4 at a constant stress of 10 Pa.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

“Film former” or “film forming agent” or “film forming resin” as used herein means a polymer which, after dissolution in at least one solvent (such as, for example, water and organic solvents), leaves a film on the substrate to which it is applied, for example, once the at least one solvent evaporates, absorbs and/or dissipates on the substrate.

“Tackiness”, as used herein, refers to the adhesion between two substances. For example, the more tackiness there is between two substances, the more adhesion there is between the substances.

“Keratinous substrates”, as used herein, include but are not limited to, skin, hair and nails.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

As defined herein, stability is tested by placing the composition in a controlled environment chamber for 8 weeks at 25° C. In this test, the physical condition of the sample is inspected as it is placed in the chamber. The sample is then inspected again at 24 hours, 3 days, 1 week, 2 weeks, weeks and 8 weeks. At each inspection, the sample is examined for abnormalities in the composition such as phase separation if the composition is in the form of an emulsion, bending or leaning if the composition is in stick form, melting, or syneresis (or sweating). The stability is further tested by repeating the 8-week test at 37° C., 40° C., 45° C., 50° C., and under freeze-thaw conditions. A composition is considered to lack stability if in any of these tests an abnormality that impedes functioning of the composition is observed. The skilled artisan will readily recognize an abnormality that impedes functioning of a composition based on the intended application.

“Volatile”, as used herein, means having a flash point of less than about 100° C.

“Non-volatile”, as used herein, means having a flash point of greater than about 100° C.

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% to 15% of the indicated number.

“Waterproof” as used herein refers to the ability to repel water and permanence with respect to water. Waterproof properties may be evaluated by any method known in the art for evaluating such properties. For example, a mascara composition may be applied to false eyelashes, which may then be placed in water for a certain amount of time, such as, for example, 20 minutes. Upon expiration of the pre-ascertained amount of time, the false eyelashes may be removed from the water and passed over a material, such as, for example, a sheet of paper. The extent of residue left on the material may then be evaluated and compared with other compositions, such as, for example, commercially available compositions. Similarly, for example, a composition may be applied to skin, and the skin may be submerged in water for a certain amount of time. The amount of composition remaining on the skin after the pre-ascertained amount of time may then be evaluated and compared. For example, a composition may be waterproof if a majority of the product is left on the wearer, e.g., eyelashes, skin, etc. In a preferred embodiment of the present invention, little or no composition is transferred from the wearer.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions.

“Transfer resistance” as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a “kiss” test. The “kiss” test may involve application of the composition to human keratin material such as hair, skin or lips followed by rubbing a material, for example, a sheet of paper, against the hair, skin or lips after expiration of a certain amount of time following application, such as 2 minutes after application. Similarly, transfer resistance of a composition may be evaluated by the amount of product transferred from a wearer to any other substrate, such as transfer from the hair, skin or lips of an individual to a collar when putting on clothing after the expiration of a certain amount of time following application of the composition to the hair, skin or lips. The amount of composition transferred to the substrate (e.g., collar, or paper) may then be evaluated and compared. For example, a composition may be transfer resistant if a majority of the product is left on the wearer's hair, skin or lips. Further, the amount transferred may be compared with that transferred by other compositions, such as commercially available compositions. In a preferred embodiment of the present invention, little or no composition is transferred to the substrate from the hair, skin or lips.

Polyamine Compound

According to the present invention, compositions comprising at least one polyamine compound are provided. In accordance with the present invention, the polyamine compound has at least two primary amine groups available to react with hydrophilic groups of the oil-soluble polar modified polymer.

According to particularly preferred embodiments, the polyamine compound is a polyalkyleneimine, preferably a C2-C5 polyalkyleneamine compound, more preferably a polyethyleneimine or polypropyleneimine. Most preferably, the polyalkylenamine is polyethyleneimine (“PEI”). The polyalkyleneamine compound preferably has an average molecular weight range of from 500-200,000, including all ranges and subranges therebetween.

According to preferred embodiments, compositions of the present invention contain polyethyleneimine compounds in the form of branched polymers. Commercially available examples of such polymers are available from BASF under the tradename LUPASOL or POLYIMIN. Non-limiting examples of such polyethyleneimines include Lupasol® PS, Lupasol® PL, Lupasol® PR8515, Lupasol® G20, Lupasol® G35.

According to other embodiments of the present invention, polyamines such as polyethyleneimines and polypropyleneimines can be in the form of dendrimers. Non-limiting examples of such dendrimers are manufactured by the company DSM, and/or are disclosed in U.S. Pat. No. 5,530,092 and U.S. Pat. No. 5,610,268, the contents of which are hereby incorporated by reference. Commercially available examples of such polymers include polyamidoamine or polypropyleneimine polymers from DENDRITECH sold under the STARBURST® name.

According to other embodiments of the present invention, derivatives of polyalkyleneamines are suitable polyamines. Such derivatives include, but are not limited to, alkylated derivatives, the addition products of alkylcarboxylic acids to polyalkyleneamines, the addition products of ketones and of aldehydes to polyalkyleneamines, the addition products of isocyanates and of isothiocyanates to polyalkyleneamines, the addition products of alkylene oxide or of polyalkylene oxide block polymers to polyalkyleneamines, quaternized derivatives of polyalkyleneamines, the addition products of a silicone to polyalkyleneamines, and copolymers of dicarboxylic acid and polyalkyleneamines. Even further suitable polyamines include, but are not limited to, polyvinylimidazoles (homopolymers or copolymers), polyvinylpyridines (homopolymers or copolymers), compounds comprising vinylimidazole monomers (see, for example, U.S. Pat. No. 5,677,384, hereby incorporated by reference), and polymers based on amino acids containing a basic side chain (preferably selected from proteins and peptides comprising at least 5%, preferably at least 10% of amino acids selected from histidine, lysine and arginine). Such suitable polyamines as described above include those disclosed and described in U.S. Pat. No. 6,162,448, the contents of which are hereby incorporated by reference. Commercially available examples of such polymers include polyvinylamine/formamide such as those sold under the Lupamine° name by BASF, chitosan from vegetable origin such as those sold under the Kiosmetine° or Kitozyme° names, or copolymer 845 sold by ISP.

According to preferred embodiments, the at least one polyamine compound is present in the composition of the present invention in an amount ranging from about 0.1 to less than 10% by weight, more preferably from about 0.2 to about 5% by weight, based on the total weight of the composition, including all ranges and subranges within these ranges. Preferably, the amount of polyamine compound reacted with the oil-soluble polar modified polymer is such that at least two amine groups on the polyamine compound react with the oil-soluble polar modified polymer to form links or bonds between the amine groups and the hydrophilic groups of the oil-soluble polar modified polymer. The appropriate amount of polyamine compound to react with the oil-soluble polar modified polymer to obtain a reaction product can be easily determined, taking into account the number/amount of reactive amine groups on the polyamine compound and the number/amount of corresponding reactive groups on the oil-soluble polar modified polymer (for example, maleic anhydride groups). According to preferred embodiments, excess oil-soluble polar modified polymer (as determined by the relative number/amount of corresponding reactive groups on the polymer as compared to the reactive amine groups on the polyamine) is reacted with polyamine. Preferably, the polyamine to oil-soluble polar modified polymer ratio is between 0.005 and 1, preferably between 0.006 and 0.5, and preferably between 0.007 and 0.1, including all ranges and subranges therebetween.

The polyamine is typically present in the composition of the invention in an amount ranging from about 0.5 to about 10% by weight, such as from about 1 to about 8% by weight, and from about 2 to about 5% by weight, including all ranges and subranges therebetween, based on the total weight of the composition.

Oil-Soluble High Carbon Polar Modified Polymer

According to the present invention, compositions comprising at least one oil-soluble high carbon polar modified polymer are provided. “Polar modified polymer” as used herein refers to a hydrophobic homopolymer or copolymer which has been modified with hydrophilic unit(s). “Oil-soluble” as used herein means that the polar modified polymer is soluble in oil. “High carbon” means more than 20 carbon atoms.

Suitable monomers for the hydrophobic homopolymers and/or copolymers include, but are not limited to, cyclic, linear or branched, substituted or unsubstituted, C22-C40 compounds such as, C22-C28 compounds, C24-C26 compounds, C26-C28 compounds, and C30-C38 compounds, including all ranges and subranges therebetween. Preferably, the monomers are C24-26 compounds, C26-C28 compounds or C30-C38 compounds.

Suitable hydrophilic unit(s) include, but are not limited to, maleic anhydride, acrylates, alkyl acrylates such as, for example, methyl acrylate, ethyl acrylate, propyl acrylate, and butyl acrylate, and polyvinylpyrrolidone (PVP).

According to preferred embodiments, the oil-soluble high carbon polar modified polymer is a wax. Also preferably, the oil-soluble high carbon polar modified polymer wax has one or more of the following properties:

a weight-average molecular weight Mw of less than or equal to 30 000 g/mol, preferably of 500 to 10 000 g/mol and particularly preferably of 1000 to 5,000 g/mol, including all ranges and subranges therebetween;

a number-average molecular weight Mn of less than or equal to 15 000 g/mol, preferably of 500 to 12 000 g/mol and particularly preferably of 1000 to 5000 g/mol, including all ranges and subranges therebetween;

a molar mass distribution Mw/Mn in the range from 1.5 to 10, preferably from 1.5 to 5, particularly preferably from 1.5 to 3 and especially preferably from 2 to 2.5, including all ranges and subranges therebetween; and/or

a crystallinity of 8% to 60%, preferably 9% to 40%, and more preferably 10% to 30%, including all ranges and subranges therebetween, as determined by differential scanning calorimetry.

According to preferred embodiments relating to a copolymer wax, it is preferable to have, based on the total weight of the copolymer backbone, 0.1 to 30% by weight of structural units originating from the one monomer and 70.0 to 99.9% by weight of structural units originating from the other monomer.

Waxes of the present invention can be based upon homopolymers or copolymers made, for example, by the process described in EP 571 882, the entire contents of which is hereby incorporated by reference. Suitable preparation processes include, for example, suspension polymerization, solution polymerization and gas-phase polymerization of olefins in the presence of catalysts, with polymerization in the monomers also being possible.

Oil-soluble high carbon polar modified polymer wax can be produced in a known manner from the homopolymers and copolymers described above by oxidation with oxygen-containing gases, for example air, or by graft reaction with polar monomers, for example maleic acid or acrylic acid or derivatives of these acids. The polar modification of polyolefin waxes by oxidation with air is described, for example, in EP 0 890 583 A1, and the modification by grafting is described, for example, in U.S. Pat. No. 5,998,547, the entire contents of both of which are hereby incorporated by reference in their entirety.

Acceptable oil-soluble high carbon polar modified polymer waxes include, but are not limited to, homopolymers and/or copolymers of C24, C25 and/or C26 groups, copolymers C26, C27 and/or C28 groups, or copolymers of C30-C38 groups, which have been modified with hydrophilic units such as, for example, maleic anhydride, acrylate, methacrylate, polyvinylpyrrolidone (PVP), etc. Preferably, the oil-soluble high carbon polar modified polymer wax has from about 5% to about 30% hydrophilic units, more preferably from about 10% to about 25% hydrophilic units by weight with respect to the weight of the wax, including all ranges and subranges therebetween. Particularly preferred hydrophilically modified waxes are C26, C27 and/or C28 homopolymers and copolymers which have been modified with maleic anhydride units.

Particularly preferred oil-soluble high carbon polar modified polymer waxes for use in the present invention are C26-C28 alpha olefin maleic acid anhydride copolymer waxes commercially available from Clariant under the trade name LICOCARE or LICOCENE. Specific examples of such waxes include products marketed by Clariant under the LicoCare name having designations such as CM 401, which is a maleic anhydride modified wax having a Mw of 2025 and a crystallinilty of 11%, C30-C38 olefin/isopropylmaleate/maleic anhydride copolymer sold by Baker Hughes under the name Performa° V 1608, and C24-C26 alpha olefin acrylate copolymer wax commercially available from Clariant under the trade name LICOCARE CA301 LP3346 based on a polar backbone with C24-26 side chains with alternating ester and carboxylic acid groups.

According to other embodiments of the present invention, the polar modified polymer is not a wax. In accordance with these embodiments of the present invention, the polar modified polymer is based upon a homopolymer and/or copolymer of hydrophobic monomer(s) and has a weight-average molecular weight Mw of less than or equal to 1,000,000 g/mol, preferably of 1000 to 250,000 g/mol and particularly preferably of 5,000 to 50,000 g/mol, including all ranges and subranges therebetween.

In accordance with these embodiments, the polar modified polymer can be of any form typically associated with polymers such as, for example, block copolymer, a grafted copolymer or an alternating copolymer. For example, the polar modified polymer can contain a hydrophobic backbone (such as polypropylene and/or polyethylene) onto which hydrophilic groups (such as maleic anhydride) have been attached by any means including, for example, grafting. The attached groups can have any orientation (for example, atactic, isotactic or syndiotactic along the backbone).

Preferably, the oil-soluble high carbon polar modified polymer(s) represent from about 1% to about 20% of the total weight of the composition, more preferably from about 3% to about 17% of the total weight of the composition, and most preferably from about 5% to about 15%, including all ranges and subranges therebetween.

Reaction Product

According to the present invention, oil soluble high carbon polar modified polymer is reacted with the polyamine compound, in the presence of water in, at minimum, an amount sufficient to solubilize the polyamine, to form a reaction product. In accordance with the present invention, the reaction product is water-insoluble.

According to preferred embodiments, the oil-soluble polar modified polymer is in an oil carrier, and the polyamine compound is in an aqueous carrier. The reaction occurs by combining the oil carrier and the aqueous carrier. Because the oil-soluble polar modified polymer is typically solid at room temperature, the oil carrier is preferably heated to liquefy the polymer prior to combination with the aqueous carrier. Preferably, the oil carrier is heated beyond the melting point of the oil-soluble polar modified polymer, typically up to about 80° C., 90° C. or 100° C. Although not wanting to be bound by any particular theory, it is believed that at a temperature below 100° C., the reaction of oil-soluble polar modified polymer with the primary amine group of the polyamine opens the anhydride ring to form a half acid and half amide crosslinked product. However, at a temperature above 100° C., the reaction of oil-soluble polar modified polymer with the primary amine group of the polyamine opens the anhydride ring to form an imide crosslinked product. The former product is preferred over the latter product. It is not necessary for all amine groups and all hydrophilic groups to react with each other to form the reaction product. Rather, it is possible that the composition may contain free polyamine and/or free oil-soluble polar modified polymer in addition to the reaction product.

Although not wanting to be bound by any particular theory, it is also believed that the polyamine(s) can be non-covalently assembled with the polar modified polymer(s) by electrostatic interaction between an amine group of the polyamine and a hydrophilic group (for example, carboxylic acid group associated with maleic anhydride groups) of the polar modified polymer to form a supramolecule. For example, with specific reference to maleic anhydride groups, in the presence of water these groups can open to form dicarboxylic acid groups which can interact with protonated primary amines of the polyamine through ionic interaction to form a polymer-polymer complex with hydrophilic core crosslinkers and a hydrophobic network that act as supramolecular capsule. If a large amount of maleic anhydride groups are present, the secondary amine groups of polyamine are also protonated and interact with alkyl carboxylates.

According to preferred embodiments, the oil-soluble polar modified polymer is in an oil carrier, and the polyamine compound is in an aqueous carrier, and the reaction occurs by combining the oil carrier and the aqueous carrier. Because the oil-soluble polar modified polymer is typically solid at room temperature, the oil carrier is preferably heated to liquefy the polymer prior to combination with the aqueous carrier. Preferably, the oil carrier is heated beyond the melting point of the oil-soluble polar modified polymer, typically up to about 80° C., 90° C. or 100° C.

Without intending to be bound by any particular theory, it is believed that the reason for this is that due to the chemical and physical reactions which take place when the oil-soluble polar modified polymer is combined with the polyamine, the subsequent reaction product that is formed is surprisingly and unexpectedly able to entrap large amounts of water molecules within its hydrophobic matrix. The resultant product is eminently capable of forming a film, is self-emulsifying, waterproof. Moreover, the product is both stable and capable of carrying various types of ingredients.

Water

The composition of the present invention also comprises water in order to both solubilize the polyamine and vary the viscoelastic properties of the composition. Further, the water is employed in an amount sufficient to achieve the targeted texture and feel. The water may be employed in an amount of from about 0.5% to about 50% by weight, such as from about 5% to about 45% by weight, such as from about 10% to about 35% by weight, such as from about 15 to about 30% by weight, including all ranges and subranges therebetween, all weights based on the total weight of the composition.

Non-Volatile Solvent

The cosmetic composition of the present invention also comprises of at least one non-volatile solvent capable of solubilizing the polar modified polymer. As used herein, the term “non-volatile” means having a flash point of greater than about 100° C. The at least one non-volatile solvent typically comprises at least one non-volatile oil. Examples of non-volatile oils that may be used in the present invention include, but are not limited to, polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≧10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C₈ to C₂₆ fatty alcohols, for instance oleyl alcohol; and
mixtures thereof.

Further, examples of non-volatile oils that may be used in the present invention include, but are not limited to, non-polar oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

Preferably, the non-volatile solvent is present in the cosmetic composition of the invention in an amount of from about 1% to about 90% by weight, such as from about 3% to about 80% by weight, such as from about 5% to about 60% by weight, including all ranges and subranges therebetween, all weights based on the total weight of the composition.

Volatile Solvents Other than Water

The compositions of the present invention preferably but optionally further comprise at least one volatile solvent. The at least one volatile solvent may be chosen from a volatile silicone oil or a volatile non-silicone oil.

Suitable volatile silicone oils include, but are not limited to, linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethyleyelopentasiloxane | 72 | 4.2 |
| (cyclopentasiloxane or D5) | | |
| Octamethyleyelotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |
| PDMS DC 200 (3 St) from Dow Corning | 102 | 3 |

Suitable volatile non-silicone oils may be selected from volatile hydrocarbon oils, alcohols, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, isohexadecane, and for example, the oils sold under the trade names of Isopar or Permethyl, the C₈ to C₁₆ branched esters such as isohexyl or isodecyl neopentanoate and their mixtures. Preferably, the volatile non-silicone oils have a flash point of at least 40° C.

Non-limiting examples of volatile non-silicone oils are listed in Table 2 below.

TABLE 2

| Compound | Flash Point (° C.) |
|---|---|
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C11-C13) | 62 |
| Isopar H (isoparaffin C11-C12) | 56 |

In general, the at least one volatile solvent, if present is preferably present in the composition in an amount of from about 20 to about 90% by weight, such as from about 30 to about 80% by weight, and from about 35 to about 75% by weight, including all ranges and subranges therebetween, all weights based on the total weight of the composition.

Optional Ingredients

The composition of the present invention may also include any one, or more, optional ingredients. Examples thereof include, but are not limited to, colorants such as pigments and dyestuffs, co-solvents, waxes, plasticizers, preservatives, fillers, active ingredients and sunscreens.

It has surprisingly been discovered that the composition of the present invention, in order to be effective as a base/matrix for carrying cosmetic ingredients, does not require the use of silicone film formers, emulsifiers or gelling agents. Without intending to be bound by theory, it is believed that the reason for this is due to the chemical and physical reactions which take place when the oil soluble high carbon polar modified polymer is combined with the polyamine. The resultant product is capable of forming a film, is self-emulsifying, waterproof, and inherently possesses a desirable cushiony/bouncy texture and feel in the absence of silicone elastomers. Moreover, the product is both stable and capable of carrying various types of cosmetic ingredients.

The present invention is further described in terms of the following non-limiting examples. Unless otherwise indicated, all parts and percentages are on a weight-by-weight percentage basis.

The composition of the present invention may be used for any application in which it is desirable to employ a waterproof film, capable of carrying cosmetic ingredients such as, for example, pigments, and which is stable and possesses a cushiony/bouncy texture and feel. One example thereof is a foundation for the face.

The present invention is further described in terms of the following non-limiting examples. Unless otherwise indicated, all parts and percentages are on a weight-by-weight percentage basis.

It has surprisingly been discovered that the composition of the present invention, in order to be effective as a base/matrix for carrying insoluble ingredients, does not require the use of silicone resins, emulsifiers or gelling agents. Accordingly, according to preferred embodiments of the present invention, the compositions of the present invention are free of any combination of silicone resins, emulsifiers, surfactants and/or gelling agents.

The compositions of the present invention also possess a texture and feel ranging from liquid to rigid gel, depending on the amount of water present in the composition and/or temperature at which the oil-soluble polar modified polymer and polyamine are reacted.

Further, the composition of the present invention possess a texture and feel ranging from liquid to rigid gel, depending on either the crosslink density of the composition which, as was indicated above, can be varied depending on the amounts of polyamine and oil-soluble polar modified polymer present in the composition or the temperature at which the reaction product is formed.

The cushiness/bounciness of the composition is characterized by its “instantaneous creep strain oscillation” which is determined over a short interval of deformation of the material or composition during a creep experiment.

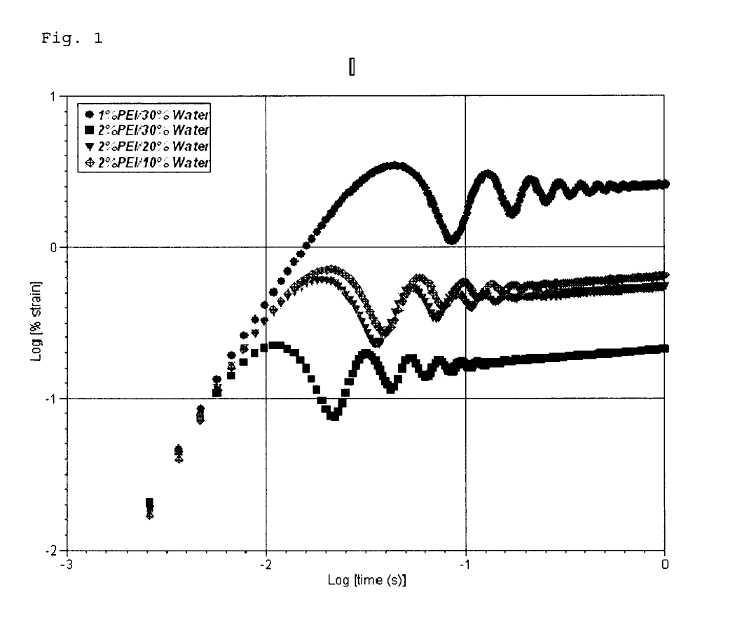

Instantaneous creep strain oscillation, over a short interval of deformation, is determined by measuring the response of the composition or material to an applied stress over a period of time of less than or equal to 1 second (t<1 second) such as, for example, from 0.001 second to 1 second. The composition or material will show some strain damping oscillations if the material is a cushiony/bouncy liquid or gel or solid, as is seen in FIG. 1.

In the event that the composition contains a low amount of water, the composition will have a soft feel and be very cushiony/bouncy. This phenomenon is evidenced by the instantaneous creep strain oscillation where a higher strain amplitude and damp are observed as compared to a material which contains larger amounts of water and, consequently, a harder feel. For a more bouncy and cushiony composition, the maximum amplitude strain of the first instantaneous oscillating peak should be larger than that of the less bouncy and less cushiony composition.

In the event the composition contains less than about 25% by weight, based on the weight of the composition, of water, it will possess a maximum instantaneous creep strain oscillation of about 0.5-50%, which translates into the material having a higher degree of cushioniness/bounciness, i.e. softer feel and texture. Conversely, if the composition contains greater than about 25% by weight, based on the weight of the composition, of water, it will possess a maximum instantaneous creep strain oscillation of 0.01-0.5%, which translates into the material having a lower degree of cushioniness/bounciness, i.e. harder feel and texture.

In the event that the composition contains less than about 10% of the oil-soluble polar modified polymer and less than about 1% of the polyamine, the composition will have a soft feel and cushiony/bouncy texture due to a low crosslink density. This phenomenon is evidenced by the instantaneous creep strain oscillation where a higher strain amplitude and damp, with higher numbers of oscillation. Thus, for those compositions in which the texture is more bouncy/cushiony, the maximum amplitude strain of the first instantaneous oscillating peak should be larger than that of the less bouncy/cushiony composition.

In the event that the composition contains more than about 10% of the oil-soluble polar modified polymer and more than about 2% of the polyamine the composition will have a harder feel and less of a cushiony/bouncy texture due to a higher crosslink density. This phenomenon is evidenced by the instantaneous creep strain oscillation where a lower strain amplitude and damp, with smaller numbers of oscillation. Thus, for a less bouncy/cushiony composition, the maximum amplitude strain of the first instantaneous oscillating peak should be smaller than that of the more bouncy/cushiony composition.

The composition of the present invention will possess, depending on the respective amounts of polyamine and oil-soluble polar modified polymer present in the composition, a maximum instantaneous creep strain oscillation of from about 0.1% to about 50% at a constant stress of 10 Pa. Varying degrees of rigidity of the material, i.e., from liquid to rigid gel, are achieved by varying the amounts of polyamine and oil-soluble polar modified polymer in the composition.

Creep Experiments

The instantaneous creep strain oscillation, creep compliance and recovery compliance are determined by using a controlled stress rheometer, commercially available from TA Instruments under the name AR-G2. The samples are measured using a parallel plate having a stainless steel, cross hatched, 40 mm diameter plate. The gap is set at 1,000 microns. The desired temperature is precisely controlled by a Peltier system.

The sample is transferred to the rheometer, and held at 25° C. The sample is measured in the creep mode at a constant stress σ₀ of 10.0 Pa, at which the creep compliance J(t) is defined as:

$$J ( t ) = \frac{γ ( t )}{{σ}_{0}}$$

Where γ(t) is the creep strain for a creep duration time t under a constant stress σ₀.

The composition of the present invention may be used for any application in which it is desirable to employ a waterproof film, capable of carrying insoluble ingredients such as, for example, pigments, and which is stable, easily spreadable, and comfortable to apply.

The present invention is further described in terms of the following non-limiting examples. Unless otherwise indicated, all parts and percentages are on a weight-by-weight percentage basis.

Based on the below examples, it was found that the composition of Example 4 was softest and most bouncy. Further, the composition of Example 3 was hardest and least bouncy, while the compositions of Examples 1 and 2 were softer and bouncier than gel of example 3.

### Examples 1-4

| INCI NAME | EX 1 | EX 2 | EX 3 | EX 4 |
|---|---|---|---|---|
| Octyldodecyl neopentanoate | 39.00 | 34.00 | 29.00 | 29.5 |
| Hydrogenated Polydecene | 39.00 | 34.00 | 29.00 | 29.5 |
| CM401* | 10.00 | 10.00 | 10.00 | 10.00 |
| Lupasol G 35 PEI | 2.00 | 2.00 | 2.00 | 1.00 |
| (PolyEthyleneImine) | | | | |
| (50% SOLID/50% WATER) | | | | |
| DI water | 10.00 | 20.00 | 30.00 | 30.00 |
| Total | 100.00 | 100.00 | 100.00 | 100.00 |

*CM401 is an Alpha olefin hydrocarbon - maleic anhydride copolymer wax commercially available from Clariant under the tradename LICOCARE CM 401 LP 3345

| | Time of the | The first | |
|---|---|---|---|
| | first maximum | maximum | Instantaneous |
| | oscillation | oscillation | Compliance of the |
| | strain peak (second) | strain peak (%) | maximum peak (1/Pa) |
| Example 1 | 0.020 | 0.71 | 7 × 10⁻⁴ |
| Example 2 | 0.019 | 0.60 | 6 × 10⁻⁴ |
| Example 3 | 0.010 | 0.22 | 2.2 × 10⁻⁴ |
| Example 4 | 0.040 | 3.40 | 22 × 10⁻⁴ |

### Procedure:

1. In container A, all the oils were heated at 90° C., then CM401 was melted in the oils until fully dissolved.
2. The container A was transferred to Silverson for homogenizing at 9000 rpm.
3. In a separate container B, Lupasol G 35 PEI (PolyEthyleneImine), and water were mixed at temperature of 80° C.-90° C.
4. Container B was then added to Container A slowly while homogenizing. The homogenizing was at 80° C. for 20-30 minutes
5. The homogenizing speed was slow down to 2000 rpm and the batch was continued homogenizing until batch was cooled to 35° C.
6. Then the batch was poured to container at room temperature.

What is claimed is:

1. A composition comprising:
(a) a reaction product of (i) at least one polyamine and (ii) at least one oil soluble high carbon polar modified polymer;
(b) water;
(c) at least one non-volatile solvent capable of solubilizing the polar modified polymer; and
(d) optionally, at least one volatile solvent other than water; and
(e) optionally, at least one colorant.

2. The composition of claim 1 wherein (i) is a branched polyethylene imine.

3. The composition of claim 1 wherein (i) is present in an amount of from about 0.05% to about 10% by weight, based on the weight of the composition.

4. The composition of claim 1 wherein (ii) is present in an amount of from about 3% to about 20% by weight, based on the weight of the composition.

5. The composition of claim 1 wherein (b) is present in an amount of from about 0.5% to about 50% by weight, based on the weight of the composition.

6. The composition of claim 1 wherein (c) is a non-volatile oil.

7. The composition of claim 1 wherein (c) is present in an amount of from about 1 to about 90% by weight, based on the weight of the composition.

8. The composition of claim 1 wherein the composition does not require a silicone elastomer, a silicone film former, a gelling agent or an emulsifier.

9. A method of making-up a keratinous substrate comprising applying onto the substrate a composition containing:
(a) a reaction product of (i) at least one polyamine and (ii) at least one oil soluble high carbon polar modified polymer;
(b) water;
(c) at least one non-volatile solvent capable of solubilizing the polar modified polymer; and
(d) optionally, at least one volatile solvent other than water; and
(e) optionally, at least one colorant.

10. The method of claim 9 wherein (i) is a branched polyethylene imine.

11. The method of claim 9 wherein (i) is present in an amount of from about 0.05 to about 10% by weight, based on the weight of the composition.

12. The method of claim 9 wherein (ii) is present in an amount of from about 3 to about 20% by weight, based on the weight of the composition.

13. The method of claim 9 wherein (b) is present in an amount of from about 0.5 to about 50% by weight, based on the weight of the composition.

14. The method of claim 9 wherein (c) is a non-volatile oil.

15. The method of claim 9 wherein (c) is present in an amount of from about 1 to about 90% by weight, based on the weight of the composition.

16. The method of claim 9 wherein the composition does not require a silicone elastomer, a silicone film former, a gelling agent or an emulsifier.
